(19) United States
(12) Patent Application Publication
(10) Pub. No.: US 2024/0385193 A1
(43) Pub. Date: Nov. 21, 2024

(54) USE OF LECTINS TO DETERMINE MAMMAGLOBIN-A GLYCOFORMS IN BREAST CANCER

(71) Applicant: Glycanostics s.r.o., Bratislava – mestská cast Dúbravka (SK)

(72) Inventors: Jan TKAC, Bratislava – mestská cast Dúbravka (SK); Tomas BERTOK, Bratislava – mestská cast Dúbravka (SK)

(21) Appl. No.: 18/691,611

(22) PCT Filed: Sep. 14, 2022
(86) PCT No.: PCT/EP2022/075496

§ 371 Date: Mar. 13, 2024

(30) Foreign Application Priority Data
Sep. 14, 2021 (EP) 21196556.1

(51) Int. Cl. G01N 33/574

(52) U.S. Cl. CPC G01N 33/57415; G01N 2333/4724

(57) ABSTRACT

The present invention relates to a method for diagnosing whether a subject may be at risk for or may suffer from breast cancer, wherein (significantly) lower or (significantly) higher binding of a binding agent to a particular glycan structure of mammaglobin-A compared to a control sample is indicative for said subject to be at risk for or to suffer from breast cancer. The present invention further relates to a kit for performing said method for diagnosing whether a subject may be at risk for or may suffer from breast cancer, comprising a binding agent capable to bind to a glycan structure of mammaglobin-A.

The present application claims the benefit of priority of European Patent Application No. 21196556.1 filed 14 Sep. 2021, the content of which is hereby incorporated by reference it its entirety for all purposes.

The present invention relates to a method for diagnosing whether a subject may be at risk for or may suffer from breast cancer, wherein (significantly) lower or (significantly) higher binding of a binding agent to a particular glycan structure of the biomarker glycoprotein mammaglobin-A compared to a control sample is indicative for said subject to be at risk for or to suffer from breast cancer. The present invention further relates to a kit for performing said method for diagnosing whether a subject may be at risk for or may suffer from breast cancer, comprising a binding agent capable to bind to a glycan structure of mammaglobin-A.

Breast cancer (BCa) is one of the most common cancer types along with lung, colorectal and prostate (only in men), with peak incidence between 45 and 65 years of age. In 2020, there were 2,261,419 of new female BCa cases worldwide with 684,996 new deaths (5^{th} most common cause of deaths among all cancer deaths) (see Sung H, Ferlay J, Siegel R L, et al. Global Cancer Statistics 2020: GLOBOCAN Estimates of Incidence and Mortality Worldwide for 36 Cancers in 185 Countries. CA: Cancer J Clin. 2021; 71: 209-249). However, mortality rate can be much lower, if routine screening for women above 40 years of age is regularly done. In 2021, the incidence was predicted to even further increase to 18 women per 100,000 women globally (see Akram M, Iqbal M, Daniyal M, et al. Awareness and current knowledge of breast cancer. Biol Res. 2017; 50: 1-23). Today, screening and early diagnostics relies on imaging methods, such as digital mammography, hand-held or automated sonography and magnetic resonance imaging (see Schunemann H J, Lerda D, Quinn C, et al. Breast cancer screening and diagnosis: a synopsis of the European Breast Guidelines. Ann Intern Med. 2020; 172: 46-56). BCa is associated strongly with genetic (especially BRCA 1 and 2 genes mutations) and other (sex, age or race—with higher mortality rate and earlier occurrence in African Americans) risk factors (see U.S. Breast Cancer Statistics https://www.breastcancer.org/symptoms/understand_bc/statistics2021 [Aug. 8, 2021]; Yedjou C G, Sims J N, Miele L, et al. Health and racial disparity in breast cancer. Breast Cancer Metast Drug Resist. 2019: 31-49). Because the specificity and sensitivity of using CEA (carcinoembryonic antigen) and CA15-3 (cancer antigen) for BCa is low, new, more specific biomarkers need to be identified.

WO 02/053017 A2 discloses a method and a kit for determining breast cancer. Tian-Hua et al. (2016), Am J Transl Res, 8(10):4250-4264, disclose glycosylation patterns and PHA-E-associated glycoprotein profiling associated with early hepatic encephalopathy in Chinese hepatocellular carcinoma patients. Xiong et al. (2002), Journal of Chromatography B, 782(1-2):405-418, disclose the use of a lectin affinity selector in the search for unusual glycosylation in proteomics. Zehentner et al. (2004), Clinical Biochemistry, 37(4):249-257, disclose mammaglobin as a candidate diagnostic marker for breast cancer. O'Brien et al. (2004), International Journal of Cancer, 114(4):623-627, disclose the existence of mammaglobin in multiple molecular forms.

Mammaglobin-A, also known as mammaglobin-1 or secretoglobin family 2A member 2, is a secreted glycoprotein, and a product of the SCGB2A2 gene (chromosome 11, synonyms: MGB1, UGB2). It is a member of the superfamily of secretoglobins, a group of small dimeric secreted and sometimes glycosylated proteins. Mammaglobin-A itself is N-glycosylated at Asn53^{a} and Asn68^{b}. It is over-expressed in breast cancer (BCa) and mammary-gland specific (analogous to PSA, which is prostate-specific protein), making it a possible biomarker of breast cancer. Especially, the inventors of the present invention have found that investigating changes of the glycan structure of this protein offers new possibilities for diagnosing of breast cancer, which has not been described so far.

The above described disadvantages need to be overcome. The present invention therefore addresses these needs and technical objectives and provides a solution as described herein and as defined in the claims.

The present invention relates to a method for diagnosing whether a subject may be at risk for or may suffer from breast cancer, comprising
(1) contacting a sample obtained from said subject, said sample comprising mammaglobin-A as a biomarker glycoprotein, with a binding agent capable to (specifically) bind to a glycan structure of mammaglobin-A,
   wherein presence or overexpression of mammaglobin-A (e.g., at least about 1.5-fold, at least about 2-fold, or at least about 3-fold overexpression), or underexpression of mammaglobin-A (e.g., at least about 1.5-fold, at least about 2-fold, or at least about 3-fold underexpression), is indicative for being at risk for and/or for presence of breast cancer, and
   wherein said glycan structure deviates from the glycan structure of mammaglobin-A as expressed in a subject not being at risk for or suffering from breast cancer, and
(2) determining whether said binding agent bound to a glycan structure of mammaglobin-A,
wherein (significantly) lower or (significantly) higher (preferably significantly higher) binding of said binding agent to said glycan structure of mammaglobin-A compared to a control sample is indicative for said subject to be at risk for or to suffer from breast cancer.

As used herein and as generally known in the art, “glycoprotein” (or “glycosylated protein”) as used herein means a protein containing one or more N-, O-, S- or C-covalently linked carbohydrates of various types, e.g., ranging from monosaccharides to branched polysaccharides (including their modifications such as sulfo- or phospho-group attachment). N-linked glycans are carbohydrates bound to —NH₂-group of asparagine. O-linked glycans are carbohydrates bound to the —OH-group of serine, threonine, or hydroxylated amino acids. S-linked glycans are carbohydrates bound to the —SH-group of cysteine. C-linked glycans are carbohydrates bound to tryptophan via C—C bond.

The term “glycan” refers to glyco-RNA and/or to compounds consisting of monosaccharides linked glycosidically and may also refer to the carbohydrate portion of a glycoconjugate, such as a glycoprotein, glycolipid, or a proteoglycan, even if the carbohydrate is only a monosaccharide or an oligosaccharide.

In one embodiment of the present invention, said subject, which may be at risk for or may suffer from breast cancer is a human being.

As has been surprisingly found in context with the present invention, the biomarker glycoprotein as described herein, which can be indicative for being at risk for and/or for presence of breast cancer, exhibits changes in the glycan structure (any statistically relevant change(s) in the glycan structure of said biomarker glycoprotein, e.g., presence or overexpression or underexpression of said biomarker glycoprotein) if a subject may be at risk for or may suffer from breast cancer. In context with the present invention, this led to the surprising finding that particular glycan structures on mammaglobin-A deviating from the “normal” glycan structure of mammaglobin-A may be indicative for being at risk for and/or for the presence of breast cancer. In accordance with the present invention, identifying such changed glycan structures on mammaglobin-A using a suitable binding agent capable to bind such glycan structure then allows diagnosing whether a subject may be at risk for or may suffer from breast cancer.

In this context, in accordance with the present invention, it is possible to use a binding agent capable to bind to the glycan structure of mammaglobin-A in non-cancerous state, contact said binding agent to a sample according to step (1) of the method described and provided herein, and to compare the binding ability of said binding agent to the glycan structure of mammaglobin-A contained in a control sample (healthy sample). Said mammaglobin-A may have a changed glycan structure compared to the glycan structure of mammaglobin-A in non-cancerous state as described in the method provided herein, e.g. may contain more (e.g., at least about 1.5×, at least about 2×, at least about 2.5×, or at least about 3× more) or may contain less (e.g., at least about 1.5×, at least about 2×, at least about 2.5×, or at least about 3× less) mammaglobin-A as biomarker glycoprotein in cancerous state.

In one embodiment of the method of the present invention, if the binding agent binds at mammaglobin-A with a higher extent (preferably significantly higher extent, e.g. at least about 1.5×, at least about 2×, at least about 2.5×, or at least about 3× higher extent) to the glycan structure of mammaglobin-A contained in the sample of a subject, which may be at risk for or may suffer from breast cancer compared to that of the control sample, this may be indicative for said subject to be at risk for or to suffer from breast cancer.

In one embodiment of the method of the present invention, if the binding agent binds at mammaglobin-A with a lower extent (preferably significantly lower extent, e.g. at least about 1.5×, at least about 2×, at least about 2.5×, or at least about 3× lower extent) to the glycan structure of mammaglobin-A contained in the sample of a subject, which may be at risk for or may suffer from breast cancer compared to that of the control sample, this may be indicative for said subject to be at risk for or to suffer from breast cancer.

Thus, in accordance with the present invention, it is possible to use a binding agent capable to bind to the glycan structure of mammaglobin-A in cancerous state, by contacting said binding agent to a sample according to step (1) of the method described and provided herein, and to compare the binding ability of said binding agent to the glycan structure of mammaglobin-A contained in a control sample (healthy sample). Preferably, if the binding agent binds at a higher extent (preferably significantly higher extent, e.g. at least about 1.5×, at least about 2×, at least about 2.5×, or at least about 3× higher extent) to the glycan structure of mammaglobin-A contained in the sample of a subject, which may be at risk for or may suffer from breast cancer compared to that of the control sample, this may be indicative for said subject to be at risk for or to suffer from breast cancer.

In one embodiment of the present invention, the glycoprotein mammaglobin-A, also called mammaglobin-1 or secretoglobin family 2A member 2, may be the mammaglobin-A from Homo sapiens, e.g. as displayed in UniProtKB-Accession Number Q13296.

In one embodiment of the present invention, said breast cancer is characterized by being Her2-negative; estrogen receptor (ER)-negative, progesterone receptor-negative (PR) and Her2-negative (triple-negative); or estrogen receptor-positive, progesterone receptor-positive and Her2-negative.

In one embodiment of the present invention, said breast cancer comprises invasive ductal carcinoma (IDC), ductal carcinoma in situ (DCIS), lobular carcinoma in situ (LCIS), ductal carcinoma of no special type (NST) or invasive lobular carcinoma (ILC).

In accordance with the present invention, the binding agent to be employed in the method described and provided herein, which is capable to (specifically) bind to a glycan structure of mammaglobin-A as described herein can be any kind of an agent, which can bind to a glycan structure. Preferably, such binding agent is an agent, where the binding thereof to a glycan structure can be measured and quantified, e.g., either where the binding itself can be detected and measured, and/or where the glycan structure is recognised by a binding agent comprising a marker molecule, which can be detected using a suitable method.

In the context with the present invention, non-limiting examples of suitable binding agents may include lectin, anti-glycan antibody, aptamer (nucleic acid aptamers, e.g., DNA or RNA aptamer, or peptide aptamer), or boronic acid or derivatives thereof. In one embodiment of the present invention, the binding agent to be employed in the method described and provided herein is a lectin. In another example, in context with the inventive method described and provided herein, said binding agent is capable to (specifically) bind to a glycan structure terminating in N-acetylgalactosamine, linked α or β to the 3 or 6 position of galactose, or which comprises a LacNAc epitope; or said binding agent is capable to (specifically) bind to a glycan structure terminating in antennary or core fucose, α-2,3-Neu5Ac (α-2,3-linked sialic acid), α-2,6-Neu5Ac (α-2,6-linked sialic acid), α-2,8-Neu5Ac (α-2,8-linked sialic acid), sialic acid (α-2,3-Neu5Ac, α-2,6-Neu5Ac or α-2,8-Neu5Ac), N-linked tri/tetra-antennary, branched β-1,6-GlcNAc, bisecting GlcNAc or branched (LacNAc)ₙ, preferably to a glycan structure terminating in N-acetylgalactosamine linked α or β to the 3 or 6 position of galactose. The binding agent may bind to a glycan structure terminating in N-acetylgalactosamine, linked α or β to the 3 or 6 position of galactose, or which comprises a LacNAc epitope. The binding agent may be capable to (specifically) bind to a glycan structure terminating in antennary or core fucose. The binding agent may be capable to (specifically) bind to α-2,3-Neu5Ac (α-2,3-linked sialic acid). The binding agent may be capable to (specifically) bind to α-2,6-Neu5Ac (α-2,6-linked sialic acid). The binding agent may be capable to (specifically) bind to α-2,8-Neu5Ac (α-2,8-linked sialic acid). The binding agent may be capable to (specifically) bind to sialic acid (α-2,3-Neu5Ac, α-2,6-Neu5Ac or α-2,8-Neu5Ac). The binding agent may be capable to (specifically) bind to N-linked tri/tetra-antennary, branched β-1,6-GlcNAc, bisecting GlcNAc or branched (LacNAc)ₙ.

Generally, as used herein, a “binding agent” (or “recognition molecule”) as used herein includes a polypeptide (e.g., a lectin or anti-glycan antibody, or fragments thereof), which comprises one or more binding domains capable of binding to a target epitope as well as other molecules capable of binding to a glycan structure (e.g., aptamers or boronic acid and derivatives thereof). A binding agent, so to say, provides the scaffold for said one or more binding domains so that said binding domain(s) can bind/interact with a given target structure/antigen/epitope. The term “binding domain” characterizes in connection with the present invention a domain of a polypeptide, which specifically binds/interacts with a given target epitope. An “epitope” is antigenic and thus the term epitope is sometimes also referred to herein as “antigenic structure” or “antigenic determinant”. In context of the present invention, a glycan structure may serve as an antigenic structure for a binding agent, e.g., lectin, anti-glycan antibody, aptamer (nucleic acid aptamers, e.g., DNA or RNA aptamer, or peptide aptamer), or boronic acid or derivatives thereof, preferably one or more lectins and/or anti-glycan antibodies, preferably one or more lectins. Thus, the binding domain is an “antigen-interaction-site”. The term “antigen-interaction-site” defines, in accordance with the present invention, a motif of a polypeptide, which is able to specifically interact with a specific antigen or a specific group of antigens, e.g. the identical antigen in different species. Said binding/interaction is also understood to define a “specific recognition”.

The term “epitope” also refers to a site on an antigen to which the binding agent binds. Preferably, an epitope is a site on a molecule to which a binding agent, e.g. lectin, anti-glycan antibody, aptamer (nucleic acid aptamers, e.g., DNA or RNA aptamer, or peptide aptamer), or boronic acid or derivatives thereof, preferably one or more lectins and/or anti-glycan antibodies, preferably one or more lectins, will bind.

The term “aptamer” as used herein refers to nucleic acid, oligonucleotide or peptide molecules that bind to a specific target molecule. As used herein, unless specifically defined otherwise, the term “nucleic acid” or “nucleic acid molecule” is used synonymously with “oligonucleotide”, “polynucleotide”, “nucleic acid strand”, or the like, and means a polymer comprising one, two, or more nucleotides, e.g., single- or double-stranded.

The term “lectin” as used herein refers to a carbohydrate-binding protein of any type and origin, including lectins, galectins, selectins, recombinant lectins, or fragments of the foregoing, as well as fragments of glycan-binding sites attached to a scaffold. The term “lectin” as used herein also includes fragments of lectins, which are capable of binding to a glycan structure. A lectin can be highly specific for a carbohydrate moiety or carbohydrate moieties (e.g., it reacts specifically with terminal glycosidic residues of other molecules such as (a) glycan(s) of a glycoprotein (e.g., branching sugar molecules of glycoproteins, e.g., such as target polypeptides within the meaning of the present invention and biomarkers as described in Table 1 herein). Lectins are commonly known in the art. A skilled person is readily available to determine, which lectin may be used for binding a carbohydrate moiety or carbohydrate moieties of interest, e.g. a carbohydrate moiety or carbohydrate moieties of a glycan attached to a protein. Preferred lectins applied in the context of the present invention are described herein. Also included by the term “lectin” are Siglecs (sialic acid-binding immunoglobulin-like lectins). Notably, the term “lectin”, when used herein, also refers to glycan-binding antibodies. Accordingly, the term “lectin”, when used herein, encompasses lectins, Siglecs as well as glycan-binding antibodies.

Lectins as described herein and to be employed in context with the present invention can be isolated and optionally purified using conventional methods known in the art. For example, when isolated from its natural source, the lectin can be purified to homogeneity on appropriate immobilized carbohydrate matrices and eluted by proper haptens (see, Goldstein & Poretz (1986) In The lectins. Properties, functions and applications in biology and medicine (ed. Liener et al.), pp. 33-247, Academic Press, Orlando, Fla.; Rudiger (1993) In Glycosciences: Status and perspectives (ed. Gabius & Gabius), pp. 415-438, Chapman and Hall, Weinheim, Germany). Alternatively, the lectin can be produced by recombinant methods according to established methods (see Streicher & Sharon (2003) Methods Enzymol. 363: 47-77). As yet another alternative, lectins can be generated using standard peptide synthesis technology or using chemical cleavage methods well-known in the art based on the amino acid sequences of known lectins or the lectin disclosed herein (e.g., U.S. Pat. No. 9,169,327 B2). Another alternative can be artificial lectins prepared by chemical modification of any above specified lectins (see Y. W. Lu, C. W. Chien, P. C. Lin, L. D. Huang, C. Y. Chen, S. W. Wu, C. L. Han, K. H. Khoo, C. C. Lin, Y. J. Chen, BAD-Lectins: Boronic Acid-Decorated Lectins with Enhanced Binding Affinity for the Selective Enrichment of Glycoproteins, Analytical Chemistry, 85 (2013) 8268-8276.).

In the context of the present invention, in case of binding of glycans to lectins (or vice versa) the binding affinity is preferably in the range of about 10⁻¹ to 10⁻¹⁰ (K_{D}), preferably about 10⁻² to 10⁻⁸ (K_{D}), more preferably about 10⁻³ to 10⁻⁵ (K_{D}). As used herein, where the binding agent is a lectin, the term “specifically” or “specific” in context with binding of a binding agent to a glycan structure may preferably mean a binding affinity of about 10⁻² to 10⁻⁸ (K_{D}), more preferably about 10⁻³ to 10⁻⁵ (K_{D}). The methods of measuring corresponding K_{D}s for binding of glycans to lectins are known in the art and are readily available to a person skilled in the art.

In one embodiment of the present invention, the binding agent to be employed in context with the present invention may be an antibody. An “antibody” when used herein is a protein comprising one or more polypeptides (comprising one or more binding domains, preferably antigen binding domains) substantially or partially encoded by immunoglobulin genes or fragments of immunoglobulin genes. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes.

In particular, an “antibody” when used herein, is typically a tetrameric glycosylated protein composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. An IgM antibody consists of 5 of the basic heterotetramer unit along with an additional polypeptide called a J chain, and contains 10 antigen binding sites, while IgA antibodies comprise from 2-5 of the basic 4-chain units, which can polymerize to form polyvalent assemblages in combination with the J-chain. In the case of IgGs, the 4-chain unit is generally about 150,000 Daltons.

Each light chain includes an N-terminal variable (V) domain (VL) and a constant (C) domain (CL). Each heavy chain includes an N-terminal V-domain (VH), three or four C-domains (CHs), and a hinge region. The constant domains are not involved directly in binding an antibody to an antigen.

The pairing of a VH and VL together forms a single antigen-binding site. The CH domain most proximal to VH is designated as CH1. Each L-chain is linked to an H-chain by one covalent disulfide bond, while the two H-chains are linked to each other by one or more disulfide bonds depending on the H-chain isotype. The VH and VL-domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3.

The term “variable” refers to the portions of the immunoglobulin domains that exhibit variability in their sequence and that are involved in determining the specificity and binding affinity of a particular antibody (i.e. the “variable domain(s)”). Variability is not evenly distributed throughout the variable domains of antibodies; it is concentrated in sub-domains of each of the heavy and light chain variable regions. These sub-domains are called “hypervariable” regions or “complementarity determining regions” (CDRs). The more conserved (i.e. non-hypervariable) portions of the variable domains are called the “framework” regions (FRM). The variable domains of naturally occurring heavy and light chains each comprise four FRM regions, largely adopting a p-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (after Chothia et al., J Mol Biol (1987), 196: 901; and MacCallum et al., J Mol Biol (1996), 262: 732). The constant domains are not directly involved in antigen binding, but exhibit various effector functions, such as, for example, antibody-dependent, cell-mediated cytotoxicity and complement activation.

The terms “CDR”, and its plural “CDRs”, refer to a complementarity determining region (CDR) of which three make up the binding character of a light chain variable region (CDRL1, CDRL2 and CDRL3) and three make up the binding character of a heavy chain variable region (CDRH1, CDRH2 and CDRH3). CDRs contribute to the functional activity of an antibody molecule and are separated by amino acid sequences that comprise scaffolding or framework regions. The exact definitional CDR boundaries and lengths are subject to different classification and numbering systems. Despite differing boundaries, each of these systems has some degree of overlap in what constitutes the so called “hypervariable regions” within the variable sequences. CDR definitions according to these systems may therefore differ in length and boundary areas with respect to the adjacent framework region (see, for example, Kabat, Chothia, and/or MacCallum; Chothia et al., J Mol Biol (1987), 196: 901; and MacCallum et al., J Mol Biol (1996), 262: 732).

The term “amino acid” or “amino acid residue” as used herein typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gin or Q); glutamic acid (Glu or E); glycine (Gly or G); histidine (His or H); isoleucine (lie or 1); leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a non-polar side chain (e.g., Ala, Cys, lie, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gin, Gly, His, Met, Phe, Ser, Thr, Trp, and Tyr).

The term “framework region” refers to the art-recognized portions of an antibody variable region that exist between the more divergent (i.e. hypervariable) CDRs. Such framework regions are typically referred to as frameworks 1 through 4 (FR1, FR2, FR3, and FR4) and provide a scaffold for the presentation of the six CDRs (three from the heavy chain and three from the light chain) in three dimensional space, to form an antigen-binding surface.

When used herein, the term “antibody” does not only refer to an immunoglobulin (or intact antibody), but also to a fragment thereof, and encompasses any polypeptide comprising an antigen-binding fragment or an antigen-binding domain. Preferably, the fragment such as Fab, F(ab′)₂, Fv, scFv, Fd, dAb, and other antibody fragments that retain antigen-binding function. Typically, such fragments would comprise an antigen-binding domain and have the same properties as the antibodies described herein.

The term “antibody” as used herein includes antibodies that compete for binding to the same epitope as the epitope bound by the antibodies of the present invention, preferably obtainable by the methods for the generation of an antibody as described herein elsewhere.

To determine if a test antibody can compete for binding to the same epitope, a cross-blocking assay e.g., a competitive ELISA assay can be performed. In an exemplary competitive ELISA assay, epitope-coated wells of a microtiter plate, or epitope-coated sepharose beads, are pre-incubated with or without candidate competing antibody and then a biotin-labeled antibody of the invention is added. The amount of labeled antibody bound to the epitope in the wells or on the beads is measured using avidin-peroxidase conjugate and appropriate substrate.

Alternatively, the antibody can be labeled, e.g., with a radioactive, an enzymatic or fluorescent label or some other detectable and measurable label. The amount of labeled antibody that binds to the antigen will have an inverse correlation to the ability of the candidate competing antibody (test antibody) to compete for binding to the same epitope on the antigen, i.e. the greater the affinity of the test antibody for the same epitope, the less labeled antibody will be bound to the antigen-coated wells.

A candidate competing antibody is considered an antibody that binds substantially to the same epitope or that competes for binding to the same epitope as an antibody of the invention if the candidate competing antibody can block binding of the antibody by at least 20%, preferably by at least 20-50%, even more preferably, by at least 50% as compared to a control performed in parallel in the absence of the candidate competing antibody (but may be in the presence of a known non-competing antibody). It will be understood that variations of this assay can be performed to arrive at the same quantitative value.

The term “antibody” also includes, but is not limited to, polyclonal, monoclonal, monospecific, polyspecific such as bispecific, non-specific, humanized, human, single-chain, chimeric, synthetic, recombinant, hybrid, mutated, grafted, and in vitro generated antibodies, with a polyclonal antibody being preferred. Said term also includes domain antibodies (dAbs) and nanobodies.

Accordingly, the term “antibody” also relates to a purified serum, i.e. a purified polyclonal serum. Accordingly, said term preferably relates to a serum, more preferably a polyclonal serum and most preferably to a purified (polyclonal) serum. The antibody/serum is obtainable, and preferably obtained, for example, by the method or use described herein.

“Polyclonal antibodies” or “polyclonal antisera” refer to immune serum containing a mixture of antibodies specific for one (monovalent or specific antisera) or more (polyvalent antisera) antigens, which may be prepared from the blood of animals immunized with the antigen or antigens.

Furthermore, the term “antibody” as employed in the invention also relates to derivatives or variants of the antibodies described herein, which display the same specificity as the described antibodies. Examples of “antibody variants” include humanized variants of non-human antibodies, “affinity matured” antibodies (see, e.g., Hawkins et al., J Mol Biol (1992), 254, 889-896; and Lowman et al., Biochemistry (1991), 30: 10832-10837) and antibody mutants with altered effector function(s) (see, e.g., U.S. Pat. No. 5,648,260).

The terms “antigen-binding domain”, “antigen-binding fragment” and “antibody binding region”, when used herein, refer to a part of an antibody molecule that comprises amino acids responsible for the specific binding between antibody and antigen. The part of the antigen that is specifically recognized and bound by the antibody is referred to as the “epitope” as described herein above. As mentioned above, an antigen-binding domain may typically comprise an antibody light chain variable region (VL) and an antibody heavy chain variable region (VH); however, it does not have to comprise both. Fd fragments, for example, have two VH regions and often retain some antigen-binding function of the intact antigen-binding domain. Examples of antigen-binding fragments of an antibody include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) a Fd fragment having the two VH and CH1-domains; (4) a Fv fragment having the VL and VH-domains of a single arm of an antibody, (5) a dAb fragment (see Ward et al., (1989) Nature 341:544-546), which has a VH-domain; (6) an isolated complementarity determining region (CDR), and (7) a single chain Fv (scFv). Although the two domains of the Fv fragment, VL and VH are coded for by separate genes, they can be joined, using recombinant methods, by a synthetic linker that enables them to be made as a single protein chain in which the VL- and VH-regions pair to form monovalent molecules (known as single chain Fv (scFv); see e.g., Bird et al., (1988) Science (1988), 242: 423-426; and Huston et al., (1988) PNAS USA (1988), 85: 5879-5883). These antibody fragments are obtained using conventional techniques known to those with skill in the art, and the fragments are evaluated for function in the same manner as are intact antibodies.

The term “monoclonal antibody” as used herein comprises chemically modified monoclonal antibodies or fragments thereof, as well as an antibody obtained from a population of substantially homogeneous antibodies, i.e. the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature (1975), 256: 495, or may be made by recombinant DNA methods (see, e.g., U.S. Pat. No. 4,816,567). The “monoclonal antibodies” may also be isolated from phage antibody libraries using the techniques described in Clackson et al., Nature (1991), 352: 624-628; and Marks et al., J Mol Biol (1991), 222: 581-597, for example.

The monoclonal antibodies herein specifically include “chimeric” antibodies (immune-globulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., PNAS USA (1984), 81: 6851-6855). Chimeric antibodies of interest herein include “primitized” antibodies comprising variable domain antigen-binding sequences derived from a non-human primate (e.g., Old World Monkey, Ape etc.) and human constant region sequences.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) of mostly human sequences, which contain minimal sequence derived from non-human immunoglobulin. For the most part, humanized antibodies are human immunoglobulins (recipient antibody) in which residues from a hypervariable region (also CDR) of the recipient are replaced by residues from a hypervariable region of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues, which are found neither in the recipient antibody nor the donor antibody. These modifications are made to further refine and optimize antibody performance. The humanized antibody optimally also will comprise at least a portion of an immunoglobulin constant region (Fc), typically that of a human immunoglobulin. For further details, see Jones et al., Nature (1986), 321: 522-525; Reichmann et al., Nature (1988), 332: 323-329; and Presta, Curr. Op. Struct. Biol. (1992), 2: 593-596.

The term “human antibody” includes antibodies having variable and constant regions corresponding substantially to human germline immunoglobulin sequences known in the art, including, for example, those described by Kabat et al. (see Kabat et al., loc. cit.). The human antibodies of the present invention may include amino acid residues not encoded by human germline immunoglobulin sequences (e.g., mutations introduced by random or site-specific mutagenesis in vitro or by somatic mutation in vivo), for example, in the CDRs, and in particular, CDR3. The human antibody can have at least one, two, three, four, five, or more positions replaced with an amino acid residue that is not encoded by the human germline immunoglobulin sequence.

As used herein, “in vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term thus preferably excludes sequences generated by genomic rearrangement in an immune cell.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments (see, e.g., Songsivilai & Lachmann, Clin Exp Immunol (1990), 79: 315-321; Kostelny et al., J Immunol (1992), 148: 1547-1553). In one embodiment, the bispecific antibody comprises a first binding domain polypeptide, such as a Fab′ fragment, linked via an immunoglobulin constant region to a second binding domain polypeptide.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas (see e.g., Kohler and Milstein, Nature (1975), 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in U.S. Pat. No. 5,223,409; Smith, Science (1985), 228: 1315-1317; Clackson et al., Nature (1991), 352: 624-628; Marks et al., J Mol Biol (1991), 222: 581-597; WO 92/18619; WO 91/17271; WO 92/20791; WO 92/15679; WO 93/01288; WO 92/01047; WO 92/09690; and WO 90/02809.

In another embodiment, a monoclonal antibody is obtained from the non-human animal, and then modified, e.g., humanized, de-immunized, chimeric, may be produced using recombinant DNA techniques known in the art. A variety of approaches for making chimeric antibodies have been described (see, e.g., Morrison et al., PNAS USA (1985), 81: 6851; Takeda et al., Nature (1985), 314: 452; U.S. Pat. Nos. 4,816,567; 4,816,397; EP 171496; EP 173494, and GB 2177096). Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison, Science (1985), 229: 1202-1207; Oi et al., BioTechniques (1986), 4: 214; U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or back-mutations. Such altered immunoglobulin molecules can be made by any of several techniques known in the art, (e.g., Teng et al., PNAS USA (1983), 80: 7308-731; Kozbor et al., Immunology Today (1983), 4: 7279; Olsson et al., Meth Enzymol (1982), 92: 3-16), and may be made according to the teachings of WO 92/06193 or EP 239400.

In case of an antibody, specific binding is believed to be effected by specific motifs in the amino acid sequence of the binding domain and the antigen and the antibody bind to each other as a result of their primary, secondary or tertiary structure as well as the result of secondary modifications of said structure. The specific interaction of the antigen-interaction-site with its specific antigen may result as well in a simple binding of said site to the antigen. Moreover, the specific interaction of the antigen-interaction-site with its specific antigen may alternatively result in the initiation of a signal, e.g. due to the induction of a change of the conformation of the antigen, an oligomerization of the antigen, etc. One example of a binding domain in line with the present invention is an anti-glycan antibody. In this context, where the binding agent is an antibody, binding may be considered “specific” when the binding affinity is higher than 10⁻¹ M. Preferably, binding is considered specific when binding affinity is about 10⁻⁵ to 10⁻¹² M (K_{D}), preferably of about 10⁻⁸ to 10⁻¹² M (where the binding agent is an antibody). If necessary, non-specific binding can be reduced without substantially affecting specific binding by varying the binding conditions. Whether the recognition molecule specifically reacts as defined herein above can easily be tested, inter alia, by comparing the reaction of said recognition molecule with an epitope with the reaction of said recognition molecule with (an) other protein(s).

In accordance with the present invention, the biomarker glycoprotein mammaglobin-A (also referred to herein as biomarker, or biomarker protein) whose presence or overexpression (e.g., at least about 1.5-fold, at least about 2-fold, or at least about 3-fold overexpression) or underexpression (e.g., at least about 1.5-fold, at least about 2-fold, or at least about 3-fold underexpression) is indicative for being at risk for and/or for presence of breast cancer may be mammaglobin-A that is present or overexpressed (e.g., at least 1.5-fold, 2-fold, or 3-fold overexpressed) or underexpressed (e.g., at least 1.5-fold, 2-fold, or 3-fold underexpressed) in a cell of a (human) subject being at risk of developing or suffering from breast cancer compared to a cell of a (human) subject not being at risk of developing or not suffering from breast cancer. Preferably, in context with the present invention, such mammaglobin-A biomarker glycoprotein has a different glycan structure in a cancerous state compared to a non-cancerous state. Accordingly, in one embodiment of the present invention, the presence or overexpression (e.g., at least 1.5-fold, 2-fold, or 3-fold overexpression) or underexpression (e.g., at least 1.5-fold, 2-fold, or 3-fold underexpression) of the biomarker glycoprotein mammaglobin-A (also referred to herein as biomarker, or biomarker protein) is indicative for being at risk for and/or for presence of breast cancer.

As used herein, “overexpression” of a glycoprotein or protein may mean any way resulting in a higher amount of such glycoprotein or protein in a cell in a subject being at risk for or suffering from breast cancer as described herein compared to a cell in a subject not being at risk for or not suffering from breast cancer. This term also includes any statistically relevant increase in expression of the respective glycoprotein or protein. For example, in accordance with the present invention, “overexpression” may mean an increased translation or transcription rate, or an overall increased synthesis of such glycoprotein or protein, while “underexpression” may mean any statistically relevant decrease in expression of the respective glycoprotein or protein, for example a decreased translation or transcription rate, or an overall decreased synthesis of such glycoprotein or protein.

As has been found in context with the present invention, mammaglobin-A exhibits a different glycan structure in samples from subjects being at risk for or suffering from breast cancer compared to mammaglobin-A contained in samples from subjects not being at risk for or not suffering from breast cancer.

In context with the present invention, the binding agent, to be employed in the method described and provided herein, is capable to bind to a glycan structure of the biomarker glycoprotein mammaglobin-A as described herein. In one embodiment of the present invention, the binding agent (preferably a lectin) is capable of (specifically) binding to one or more of any one of or is capable of (specifically) binding to a glycan structure containing or terminating in core fucose, antennary fucose, Fuc-α-1,6-GlcNAc-N-Asn containing N-linked oligosaccharides, Fuc-α-1,6/3-GlcNAc, a-L-Fuc, Fuc-α-1,2-Gal-β-1,4(Fuc-α-1,3)GlcNAc, Fuc-α-1,2-Gal, Fuc-α-1,6-GlcNAc, Man-β-1,4-GlcNAc-β-1,4-GlcNAc, branched N-linked hexa-saccharide, Man-α-1,3-Man, a-D-Man, GlcNAc-β-1,4-Gal, Gal-β-1,4-GlcNAc, GlcNAc-α-1,4-Gal-β-1,4-GlcNAc, Neu5Ac (sialic acid), Gal-α-1,3-GalNAc, Gal-β-1,6-Gal, Gal-β-1,4-GlcNAc, Gal-β-1,3-GalNAc, GalNAc-α-1,3-GalNAc, GalNAc-α-1,3-Gal, GalNAc-α/β-1,3/4-Gal, a-GalNAc, GalNAc-β-1,4-Gal, GalNAc-α-1,3-(Fuc-α-1,2)Gal, GalNAc-α-1,2-Gal, GalNAc-α-1,3-GalNAc, GalNAc-β-1,3/4-Gal, GalNAc-β-1,4-GlcNAc (LacdiNAc), LacNAc, N-glycolyl sialic acid, α-2,3-Neu5Ac (α-2,3-linked sialic acid), α-2,6-Neu5Ac (α-2,6-linked sialic acid), α-2,8-Neu5Ac (α-2,8-linked sialic acid), sialic acid (α-2,3-Neu5Ac, α-2,6-Neu5Ac or α-2,8-Neu5Ac), N-acetylglucosamine-β-(1,2)-mannopyranosyl, Neu5Ac-α-4/9-O-Ac-Neu5Ac, Neu5Ac-α-2,3-Gal-β-1,4-Glc/GlcNAc, Neu5Ac-α-2,6-Gal/GalNAc, N-linked bi-antennary, N-linked tri/tetra-antennary, branched β-1,6-GlcNAc, Gal-α-1,3(Fuc-α-1,2)Gal-β-1,3/4-GlcNAc, Gal-β-1,3(Fuc-α-1,4)GlcNAc, NeuAc-α-2,3-Gal-β-1,3(Fuc-α-1,4)GlcNAc, Fuc-α-1,2-Gal-β-1,3(Fuc-α-1,4)GlcNAc, Gal-β-1,4(Fuc-α-1,3)GlcNAc, NeuAc-α-2,3-Gal-β-1,4(Fuc-α-1,3)GlcNAc, Fuc-α-1,2-Gal-β-1,4(Fuc-α-1,3)GlcNAc, high mannose, sialyl Lewis^{a} (sialyl Le^{a}) antigen, sialyl Lewis^{x} (sialyl Le^{x}) antigen, Lewis^{x} (Le^{x}) antigen, sialyl Tn antigen, sialyl T antigen, Lewis^{Y} (Le^{Y}) antigen, sulfated core 1 glycan, Tn antigen, T antigen, core 2 glycan, Lewis^{a} (Le^{a}) antigen, (GlcNAc-β-1,4)ₙ, β-D-GlcNAc, GalNAc, Gal-GlcNAc, GlcNAc, Gal-α-1,3-Gal, Gal-β-1,3-GalNAc, a-Gal, α-GalNAc, (GlcNAc)ₙ, β-1,6-GlcNAc, bisecting GlcNAc or branched (LacNAc)ₙ.

As described herein, in one embodiment of the present invention, the binding agent to be employed in the method described and provided herein may inter alia be capable of (specifically) binding a glycan structure terminating in N-acetylgalactosamine, linked α or β to the 3 or 6 position of galactose, or the binding agent may comprise a LacNAc epitope; or said binding agent may inter alia be capable of (specifically) binding a glycan structure terminating in antennary or core fucose, α-2,3-Neu5Ac (α-2,3-linked sialic acid), α-2,6-Neu5Ac (α-2,6-linked sialic acid), α-2,8-Neu5Ac (α-2,8-linked sialic acid), sialic acid (α-2,3-Neu5Ac, α-2,6-Neu5Ac or α-2,8-Neu5Ac), N-linked tri/tetra-antennary, branched β-1,6-GlcNAc, bisecting GlcNAc or branched (LacNAc)n, preferably binding a glycan structure terminating in N-acetylgalactosamine linked α or β to the 3 or 6 position of galactose. The binding agent may bind to a glycan structure terminating in N-acetylgalactosamine, linked α or β to the 3 or 6 position of galactose, or which comprises a LacNAc epitope. The binding agent may be capable to (specifically) bind to a glycan structure terminating in antennary or core fucose. The binding agent may be capable to (specifically) bind to α-2,3-Neu5Ac (α-2,3-linked sialic acid). The binding agent may be capable to (specifically) bind to α-2,6-Neu5Ac (α-2,6-linked sialic acid). The binding agent may be capable to (specifically) bind to α-2,8-Neu5Ac (α-2,8-linked sialic acid). The binding agent may be capable to (specifically) bind to sialic acid (α-2,3-Neu5Ac, α-2,6-Neu5Ac or α-2,8-Neu5Ac). The binding agent may be capable to (specifically) bind to N-linked tri/tetra-antennary, branched β-1,6-GlcNAc, bisecting GlcNAc or branched (LacNAc)ₙ.

In one embodiment, said binding agent binds to a glycan structure terminating in N-acetylgalactosamine, linked α or β to the 3 or 6 position of galactose, or which comprises a LacNAc epitope; or wherein said binding agent binds to a glycan structure terminating in antennary or core fucose, α-2,3-Neu5Ac (α-2,3-linked sialic acid), α-2,6-Neu5Ac (α-2,6-linked sialic acid), α-2,8-Neu5Ac (α-2,8-linked sialic acid), or sialic acid (α-2,3-Neu5Ac, α-2,6-Neu5Ac or α-2,8-Neu5Ac).

As has surprisingly been found in context with the present invention, mammaglobin-A as contained in samples from subjects being at risk for or suffering from breast cancer (“cancerous mammaglobin-A”) exhibits a different glycan structure compared to mammaglobin-A contained in samples from subjects not being at risk for or not suffering from breast cancer. In accordance with the present invention, such “cancerous mammaglobin-A” may be detected using binding agents, which are capable of binding the glycan structure of such “cancerous mammaglobin-A” as described herein. As has further been found in context with the present invention, mammaglobin-A as contained in samples from subjects being at risk for or suffering from breast cancer (“cancerous mammaglobin-A”) can be bound (and thus detected) by using specific lectins such as, e.g., Wisteria floribunda lectin (WFA/WFL). Accordingly, in one embodiment of the present invention, said binding agent to be employed in the method described and provided herein, which is capable of binding to a glycan structure of the biomarker glycoprotein mammaglobin-A as described herein, may be capable of (specifically) binding to the same glycan structure as Wisteria floribunda lectin (WFA/WFL) or PHA, preferably PHA-L, or a combination thereof with an affinity of at least about 80%, at least about 85%, at least about 90%, at least about 95%, at least about 96%, at least about 97%, at least about 98%, at least about 99%, or with 100% of the affinity with which PHA, preferably PHA-L, or WFL or a combination thereof bind(s) to said glycan structure. Methods to determine affinity levels of binding agents (e.g., lectins) to glycan structures are generally known in the art and comprise inter alia surface plasmon resonance, isothermal microcalorimetry, or ELISA and ELISA-like formats, preferably surface plasmon resonance.

In a more specific embodiment of the present invention, said binding agent to be employed in the method described and provided herein, which is capable of binding to a glycan structure of the biomarker glycoprotein mammaglobin-A as described herein, may be WFL, PHA, AAL, UEA-I, LCA, PSL, AAA, LTA, HPA, LBA, PhoSL, AOL, WA, Siglec 1, Siglec 4, Siglec 8, TJA-I, SCA, WGA, SNA, MAA II, Con A, GNA, MGL, NPA, Jacalin, DBA, Galectin 1, Galectin 3, Galectin 8, RCA I, RCA 120, Bandeiraea simplicifolia lectin I (BS-I), MGL (macrophage galactose-type lectin), P-selectin, H-selectin and E-selectin, or a combination thereof.

In another more specific embodiment of the present invention, said binding agent to be employed in the method described and provided herein, which is capable of binding to a glycan structure of the biomarker glycoprotein mammaglobin-A as described herein, may be WFL, PHA-L, AAL, UEA-I, LCA, PSL, AAA, LTA, HPA, LBA, PhoSL, AOL, VVA, Siglec 1, Siglec 4, Siglec 8, TJA-I, SCA, WGA, SNA, MAA II, Con A, GNA, MGL, NPA, Jacalin, DBA, PHA-E, Galectin 1, Galectin 3, Galectin 8, RCA I, RCA 120, Bandeiraea simplicifolia lectin I (BS-I), MGL (macrophage galactose-type lectin), P-selectin, H-selectin and E-selectin, or a combination thereof.

In a specific embodiment of the present invention, said binding agent is Wisteria floribunda lectin (WFA/WFL) or PHA, preferably PHA-L, or a combination thereof, preferably Wisteria floribunda lectin (WFA/WFL). Most preferably, said binding agent is a combination of Wisteria floribunda lectin (WFA/WFL) or PHA, preferably PHA-L.

In the context of the present invention AAA means Anguilla anguilla agglutinin (see e.g. UniProtKB Accession Number: Q7SIC1), AAL means Aleuria aurantia lectin, AOL means Aspergillus oryzae lectin, BS-I means Bandeiraea simplicifolia lectin and is also known as Griffonia (Bandeiraea) simplicifolia lectin I, Con A means Concanavalin A, DBA means Dolichos biflorus agglutinin, GNA means Galanthus nivalis agglutinin, HPA means Helix pomatia agglutinin, LBA means Phaseolus lunatus (lima bean, LBA), LCA means Lens culinaris agglutinin, LTA means Lotus tetragonolobus lectin, MAA II means Maackia amurensis agglutinin II, MGBL 1 means macrophage galactose binding lectin 1, NPA means Narcissus pseudonarcissus (Daffodil) lectin, PHA means PHA-E and/or PHA-L, PHA-E means Phaseolus vulgaris agglutinin E, PHA-L means Phaseolus vulgaris agglutinin L, PhoSL means Pholiota squarrosa lectin, PSL means Pisum sativum lectin, RCA I means Ricinus communis agglutinin I, SCA means Sambucus canadensis agglutinin, SNA means Sambucus nigra agglutinin, TJA-I means Trichosanthes japonica agglutinin I, UEA means Ulex europaeus agglutinin, VVA means Vicia villosa lectin, WFA means Wisteria floribunda lectin, WGA means wheat germ agglutinin and TVA means Triticum vulgaris agglutinin.

AAL, UEA-I, LCA, PSL, AAA, LTA, HPA, LBA, PhoSL, AOL, and VVA are capable of recognizing fucose. Siglec 1, Siglec 4, Siglec 8, TJA-I, SCA, WGA, SNA, and MAA II are capable of recognizing sialic acid. Con A, GNA, MGL, and NPA are capable of recognizing mannoses. Jacalin, DBA, and PHA-E are capable of recognizing branched structures or bisecting glycans. Galectin 1, Galectin 3, Galectin 8, RCA I, and RCA 120 are capable of recognizing galactose.

In context with the present invention, it is also possible to combine two or more binding agents to be employed in the method described and provided herein, which are capable to bind to a glycan structure of the biomarker glycoprotein mammaglobin-A as described herein.

In some instances, by combining two or more of such binding agents, diagnostic potential may be increased. In this context, in accordance with the present invention, it is possible to either use two or more binding agents (e.g., lectins) in the same assay, or—preferably—to use such two or more binding agents (e.g., lectins) in different assays (using the same sample) in step (1) of the inventive method, and then separately determine in step (2) whether each of respective said binding agents bound to a glycan structure of mammaglobin-A, and then to combine the information thus obtained for diagnosing whether a subject may be at risk for or may suffer from breast cancer. In one embodiment of the present invention, if two (or more) of such binding agents are employed in the method of the present invention, such binding agents are both lectins. In a specific embodiment in this context, if two (or more) of such binding agents are employed in the method of the present invention, such lectins are or comprise Wisteria floribunda lectin (WFA/WFL) and PHA, preferably PHA-L. In one embodiment, said binding agent comprises WFA/WFL and PHA, preferably PHA-L. In a preferred embodiment, said binding agent is a combination of WFA/WFL and PHA, preferably PHA-L.

For the method as described and provided herein, in context with the present invention, any suitable assay may be employed with which binding of the binding agent as described herein to the biomarker glycoprotein mammaglobin-A as described herein can be detected and quantified. Such suitable assays are generally known in the art and comprise, inter alia, ELISA or Western Blot (particularly, where the binding agent is an antibody), or lectin-based assays (see, e.g., assay as described in WO 2019/185515), or enzyme-linked lectin-binding assay ELLBA (on cells, CELLBA; cf., e.g., Gavérieux et al., J Immunol Methods (1987), 104(1-2): 173-182). In one embodiment of the present invention, a lectin-based assay is employed. In one preferred embodiment of the present invention, an enzyme-linked lectin-binding assay (ELLBA) or magnetic enzyme-linked lectin assay (MELLBA) is employed, preferably MELLBA.

The present invention further relates to a kit for performing the method for diagnosing whether a subject may be at risk for or may suffer from breast cancer, comprising a binding agent capable to bind to a glycan structure of said biomarker protein mammaglobin-A as described herein.

In one preferred embodiment of the kit of the present invention, said binding agent may be a lectin.

In a more preferred embodiment of the kit of the present invention, said binding agent to be employed in the method described and provided herein, which is capable to bind to a glycan structure of the biomarker glycoprotein mammaglobin-A, as described herein, may be capable of (specifically) binding to the same glycan structure as Wisteria floribunda lectin (WFA/WFL) or PHA, preferably PHA-L, with an affinity of at least about 80%, at least about 85%, at least about 90%, at least about 95%, at least about 96%, at least about 97%, at least about 98%, at least about 99%, or with 100% of the affinity with which Wisteria floribunda lectin (WFA/WFL) or PHA, preferably PHA-L, binds to said glycan structure.

In an even more specific embodiment of the kit of the present invention, said binding agent may be, e.g., WFL, PHA, AAL, UEA-I, LCA, PSL, AAA, LTA, HPA, LBA, PhoSL, AOL, VVA, Siglec 1, Siglec 4, Siglec 8, TJA-I, SCA, WGA, SNA, MAA II, Con A, GNA, MGL, NPA, Jacalin, DBA, Galectin 1, Galectin 3, Galectin 8, RCA I, RCA 120, Bandeiraea simplicifolia lectin I (BS-I), MGL (macrophage galactose-type lectin), P-selectin, H-selectin and E-selectin. In some instances, by combining two or more of such binding agents, diagnostic potential may be increased. Thus, in one embodiment of the kit of the present invention, the kit described and provided herein comprises two or more of such binding agents. In this context, in a specific embodiment of the kit of the present invention, both or at least two of such binding agents comprised by said kit are lectins. In a more specific embodiment in this context, such two or more lectins comprised by said kit are or comprise WFA/WFL and PHA, preferably PHA-L.

The kit as described and provided in context with the present invention may also comprise further suitable ingredients as readily understood by the skilled person, e.g., enzymes and buffers as needed to perform the method by employing a suitable assay as described herein (e.g., ELISA, Western Blot, lectin-based assay, ELLBA, MELLBA, or others).

The kits of the invention can be used in the methods of the invention.

The embodiments, which characterize the present invention, are described herein, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% or 2% of a given value or range and includes as well the given value.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes, when used herein, with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The present invention is further illustrated by the following examples. Yet, the examples and specific embodiments described therein must not be construed as limiting the invention to such specific embodiments.

### EXAMPLES

The methodology used herein is well-known and also published in, e.g., Mislovicová et al., Biointerfaces (2012), 94: 163-169. Polyclonal anti-mammaglobin-A antibody was immobilized on the bottom of an ELISA plate well. After a washing step, the surface was blocked (with human serum albumin) and washed again using previously optimized protocol. Subsequently (with additional washing step after each of the following steps), (i) diluted human serum samples, (ii) biotinylated lectins and (iii) streptavidin-peroxidase (from horseradish) were added to the plate to complete the sandwich configuration. A signal was generated using OPD/hydrogen peroxide system, the reaction was stopped using sulphuric acid and signal was read at 450 nm. The assay format was simplified without using magnetic beads since mammaglobin-A is present in blood at much higher concentration compared to PSA and thus mammaglobin-A does not need to be pre-enriched using magnetic beads, even though employment of magnetic beads can be considered and should generate at least as clear results.

Response toward lectin binding for individual samples (measured at least in duplicates) was evaluated using ROC (Receiver Operating Curve) curves and AUC (Area Under Curve) parameter for individual markers (mammaglobin-A level, age and individual lectins) and their combinations, respectively, using OriginPro® software and R in free version of RStudio, as previously reported (cf. Bertokova et al., Bioorganic & Medicinal Chemistry (2021), 116156; Bertok et al., Glycoconjugate Journal (2020), 37: 703-711). ROC curves were obtained for the two individual lectins PHA-L and WFL and their combination in case of complete early diagnostics (no subtypization) and HER2-subtype. AUC values were below the internal threshold (i.e. 0.8). Proposed N-glycan epitopes recognized by PHA-L and WFL lectins were used.

Real plasma samples were collected from the National Oncology Institute in Bratislava, Slovakia. However, serum samples are also possible here. The total amount of plasma samples in the study was n=52. The 52 breast cancer patient samples had the following characteristics: TNM (T₁=30, T₂=21, T₃=1) (no distant metastases), IDC=47, ILC=3, others=2) (invasive ductal/invasive lobular), HER2(−)=36, triple(−)=19, ER(+), PR(+), HER2(−)=15. 24 Controls (anonymous, non-BCa patients) were used.

Results showed that glycoprofiling of mammaglobin-A is applicable for diagnosing (early stage) BCa. The best lectin to detect (early stage) BCa was shown to be a combination of WFL and PHA-L with AUC 0.864 (Table 1) (WFL as used herein is Wisteria floribunda lectin (WFA/WFL)).

Thus, it was possible to combine two lectins in order to further enhance discrimination potential of the mammaglobin-A glycoprofiling. The best combination of two lectins was WFL and PHA-L (Table 1).

TABLE 1
Parameters (AUC value with left and right confidence intervals),
specificity, sensitivity and assay accuracy for individual WFL
marker, PHA-L marker and the combination thereof.

| | | | | | | |
|---|---|---|---|---|---|---|
| | | | | | CI95 | CI95 |
| Marker | Sensitivity | Specificity | Accuracy | AUC | left | right |

BCa cohort vs. Control group (early diagnostics)

| | | | | | | |
|---|---|---|---|---|---|---|
| PHA-L | 0.827 | 0.667 | 0.717 | 0.805 | 0.703 | 0.897 |
| WFL | 0.462 | 0.833 | 0.716 | 0.632 | 0.497 | 0.756 |
| PHA-L + | 0.962 | 0.625 | 0.731 | 0.864 | 0.768 | 0.941 |
| WFL | | | | | | |

BCa cohort vs. ER+/PR+/HER2-subgroup

| | | | | | | |
|---|---|---|---|---|---|---|
| PHA-L | 0.733 | 0.917 | 0.804 | 0.904 | 0.789 | 0.983 |
| WFL | 0.933 | 0.583 | 0.799 | 0.790 | 0.633 | 0.914 |
| PHA-L + | 0.733 | 0.917 | 0.804 | 0.889 | 0.767 | 0.972 |
| WFL | | | | | | |

1. Method for diagnosing whether a subject may be at risk for or may suffer from breast cancer, comprising
(1) contacting a sample obtained from said subject, said sample comprising mammaglobin-A as a biomarker glycoprotein, with a binding agent capable to bind to a glycan structure of mammaglobin-A,
wherein presence or overexpression of mammaglobin-A is indicative for being at risk for and/or for presence of breast cancer, and
wherein said glycan structure deviates from the glycan structure of mammaglobin-A as expressed in a subject not being at risk for or suffering from breast cancer, and
(2) determining whether said binding agent bound to a glycan structure of mammaglobin-A,
wherein lower or higher binding of said binding agent to said glycan structure of mammaglobin-A compared to a control sample is indicative for said subject to be at risk for or to suffer from breast cancer.

2. Method according to claim 1, wherein said subject is a human being.

3. Method according to claim 1 or 2, wherein said breast cancer is characterized by being Her2-negative; estrogen receptor (ER)-negative, progesterone receptor-negative (PR) and Her2-negative (triple-negative); or estrogen receptor-positive, progesterone receptor-positive and Her2-negative.

4. Method according to any one of the preceding claims, wherein said breast cancer comprises invasive ductal carcinoma (IDC), ductal carcinoma in situ (DCIS), lobular carcinoma in situ (LCIS), ductal carcinoma of no special type (NST) or invasive lobular carcinoma (ILC).

5. Method according to any one of the preceding claims, wherein said binding agent is a lectin, an anti-glycan antibody, an aptamer, or boronic acid or derivatives thereof.

6. Method according to any one of the preceding claims, wherein said binding agent binds to one or more of any one of core fucose, antennary fucose, Fuc-α-1,6-GlcNAc-N-Asn containing N-linked oligosaccharides, Fuc-α-1,6/3-GlcNAc, a-L-Fuc, Fuc-α-1,2-Gal-β-1,4(Fuc-α-1,3)GlcNAc, Fuc-α-1,2-Gal, Fuc-α-1,6-GlcNAc, Man-β-1,4-GlcNAc-β-1,4-GlcNAc, branched N-linked hexa-saccharide, Man-α-1,3-Man, a-D-Man, GlcNAc-β-1,4-Gal, Gal-β-1,4-GlcNAc, GlcNAc-α-1,4-Gal-β-1,4-GlcNAc, Neu5Ac (sialic acid), Gal-α-1,3-GalNAc, Gal-β-1,6-Gal, Gal-β-1,4-GlcNAc, Gal-β-1,3-GalNAc, GalNAc-α-1,3-GalNAc, GalNAc-α-1,3-Gal, GalNAc-α/β-1,3/4-Gal, a-GalNAc, GalNAc-β-1,4-Gal, GalNAc-α-1,3-(Fuc-α-1,2)Gal, GalNAc-α-1,2-Gal, GalNAc-α-1,3-GalNAc, GalNAc-β-1,3/4-Gal, GalNAc-β-1,4-GlcNAc (LacdiNAc), LacNAc, N-glycolyl sialic acid, α-2,3-Neu5Ac (α-2,3-linked sialic acid), α-2,6-Neu5Ac (α-2,6-linked sialic acid), α-2,8-Neu5Ac (α-2,8-linked sialic acid), sialic acid (α-2,3-Neu5Ac, α-2,6-Neu5Ac or α-2,8-Neu5Ac), N-acetylglucosamine-β-(1,2)-mannopyranosyl, Neu5Ac-α-4/9-O-Ac-Neu5Ac, Neu5Ac-α-2,3-Gal-β-1,4-Glc/GlcNAc, Neu5Ac-α-2,6-Gal/GalNAc, N-linked bi-antennary, N-linked tri/tetra-antennary, branched β-1,6-GlcNAc, Gal-α-1,3(Fuc-α-1,2)Gal-β-1,3/4-GlcNAc, Gal-β-1,3(Fuc-α-1,4)GlcNAc, NeuAc-α-2,3-Gal-β-1,3(Fuc-α-1,4)GlcNAc, Fuc-α-1,2-Gal-β-1,3(Fuc-α-1,4)GlcNAc, Gal-β-1,4(Fuc-α-1,3)GlcNAc, NeuAc-α-2,3-Gal-β-1,4(Fuc-α-1,3)GlcNAc, Fuc-α-1,2-Gal-β-1,4(Fuc-α-1,3)GlcNAc, high mannose, sialyl Lewis^{a} (sialyl Le^{a}) antigen, sialyl Lewis^{x} (sialyl Le^{x}) antigen, Lewis^{x} (Le^{x}) antigen, sialyl Tn antigen, sialyl T antigen, Lewis^{Y} (Le^{Y}) antigen, sulfated core 1 glycan, Tn antigen, T antigen, core 2 glycan, Lewis^{a} (Le^{a}) antigen, (GlcNAc-β-1,4)ₙ, B-D-GlcNAc, GalNAc, Gal-GlcNAc, GlcNAc, Gal-α-1,3-Gal, Gal-β-1,3-GalNAc, a-Gal, a-GalNAc, (GlcNAc)ₙ, β-1,6-GlcNAc, bisecting GlcNAc or branched (LacNAc)ₙ.

7. Method according to any one of the preceding claims, wherein said binding agent binds to a glycan structure terminating in N-acetylgalactosamine, linked α or β to the 3 or 6 position of galactose, or which comprises a LacNAc epitope; or wherein said binding agent binds to a glycan structure terminating in antennary or core fucose, α-2,3-Neu5Ac (α-2,3-linked sialic acid), α-2,6-Neu5Ac (α-2,6-linked sialic acid), α-2,8-Neu5Ac (α-2,8-linked sialic acid), sialic acid (α-2,3-Neu5Ac, α-2,6-Neu5Ac or α-2,8-Neu5Ac), N-linked tri/tetra-antennary, branched β-1,6-GlcNAc, bisecting GlcNAc or branched (LacNAc)ₙ.

8. Method according to any one of the preceding claims, wherein said binding agent binds to the same glycan structure as PHA or WFL or a combination thereof with an affinity of at least 80% of the affinity with which PHA or WFL or a combination thereof binds to said glycan structure.

9. Method according to any one of the preceding claims, wherein said binding agent is WFL, PHA, AAL, UEA-I, LCA, PSL, AAA, LTA, HPA, LBA, PhoSL, AOL, VVA, Siglec 1, Siglec 4, Siglec 8, TJA-I, SCA, WGA, SNA, MAA II, Con A, GNA, MGL, NPA, Jacalin, DBA, Galectin 1, Galectin 3, Galectin 8, RCA I, RCA 120, Bandeiraea simplicifolia lectin I (BS-I), MGL (macrophage galactose-type lectin), P-selectin, H-selectin and E-selectin, or a combination thereof.

10. Method according to any one of the preceding claims, wherein said binding agent is PHA or WFL or a combination thereof.

11. Method according to any one of the preceding claims, wherein a lectin-based assay is employed.

12. Method according to claim 11, wherein an enzyme-linked lectin-binding assay (ELLBA) or a magnetic enzyme-linked lectin-binding assay (MELLBA) is employed.

13. Kit for performing the method of any one of the preceding claims, comprising a binding agent capable to bind to a glycan structure of mammaglobin-A.

14. Kit according to claim 13, wherein said binding agent is a lectin.

15. Kit according to claim 13 or 14, wherein said lectin is WFL or PHA or a binding agent binding to the same glycan structure as PHA or WFL with an affinity of at least 80% of the affinity with which PHA or WFL bind to said glycan structure.

16. The method of claim 10, wherein said binding agent is a combination of PHA and WFL.

17. Use of the kit of any one of claims 13 to 15 in a method according to any one of claims 1-12 and 16.
